(19) United States
(12) Patent Application Publication
(10) Pub. No.: US 2017/0209131 A1
(43) Pub. Date: Jul. 27, 2017

(54) VASCULATURE CLOSURE DEVICES AND AUTOMATIC DEPLOYMENT SYSTEMS

(71) Applicant: E-Pacing, Inc., Wilmington, New Castle County, DE (US)

(72) Inventors: Abraham PENNER, Tel Aviv (IL); Alon BEN-YOSEF, Ramot Manasha (IL); Lone WOLINSKY, Ramat Gan (IL)

(21) Appl. No.: 15/329,107

(22) PCT Filed: Jul. 21, 2015
(86) PCT No.: PCT/US2015/041288

§ 371 Date: Jan. 25, 2017

Related U.S. Application Data
(60) Provisional application No. 62/029,008, filed on Jul. 25, 2014.

(51) Int. Cl. A61B 17/00

(52) U.S. Cl. CPC A61B 17/0057; A61B 2017/00597; A61B 2017/0061; A61B 2017/00623; A61B 2090/0811

(57) ABSTRACT

Vasculature closure device delivery systems and methods are provided. In one or more embodiments, a vasculature closure device delivery system for intraluminally positioning a vasculature closure device against a puncture site existing in a wall of a vessel includes a loading tube configured to receive the vasculature closure device therein, a push rod configured to extend through the loading tube and to push the vasculature closure device out of the loading tube and into a lumen of the vessel, and an automatic deployment mechanism configured to detect when the vasculature closure device is positioned at the puncture site and to automatically deploy the vasculature closure device thereabout.

### CROSS-REFERENCE TO RELATED APPLICATIONS

This application claims the benefit of U.S. Provisional Application No. 62/029,008, filed on Jul. 25, 2014, which is incorporated herein by reference in its entirety.

### BACKGROUND

This disclosure relates generally to the field of implantable medical devices and treatment methods, and more particularly to vasculature devices, systems, and methods for closing openings in vessel walls.

During certain types of vascular surgery, catheters are inserted through an incision in the skin and underlying tissue to access an artery, such as the femoral artery, as one example. After the surgical procedure is completed and the catheter is removed from the patient, the access hole must be closed. This is quite difficult, not only because of the high blood pressure in the artery, but also because there are many layers of tissue that must be penetrated to reach the femoral artery.

Physicians currently use a number of methods to close the artery access hole, such as localized compression, sutures, collagen plugs, adhesives, gels, foams, and/or other similar materials. To provide localized compression, the physician presses down against the vessel to allow the artery access hole to naturally clot. This method, however, can take half an hour or more, and requires the patient to remain immobilized for at least that period of time and be subsequently kept in the hospital for observation. In addition, this procedure increases the potential for clots at the puncture site to become dislodged. Moreover, the amount of time necessary for the compression can be significantly greater, depending upon how much heparin, glycoprotein IIb/IIA antagonists, or other anti-clotting agents were used during the procedure. Sutures, collagen plugs, adhesives, gels, and foams may have procedure variability, may require time to close the vessel, may have negative cost factors, may necessitate a possibly complicated deployment process, and may necessitate a separate deployment device.

For newer endovascular procedures, such as abdominal or thoracic aortic aneurysm repair, percutaneous valve replacement and repair, or cardiac ablation, which use large bore delivery systems typically in the range of 8-25 Fr, existing closure methods are suboptimal.

Certain devices, systems, and methods have been developed for closing openings in vessel walls. International Patent Application Publication No. WO 2011/046932 to Penner et al. and International Patent Application Publication No. WO 2013/188351 to Penner et al. provide various examples of vasculature closure devices and systems and methods for deploying and performing treatment using the same. These publications are incorporated herein by reference in their entirety.

There remains a need for improved vasculature closure devices and systems and methods for deploying and performing treatment using the same. It would, therefore, be advantageous to provide a vasculature closure device (VCD), system, and method that would more quickly and effectively close openings (e.g., punctures) in vessel walls. Such a device would advantageously avoid, or at least substantially reduce, the aforementioned time and expense of applying manual pressure to the opening, simplify the steps required to close the opening, avoid widening of the opening, and more effectively retain the closure device in the vessel. A more effective, safer, and easier to deliver closure device may also be beneficial for smaller sheath accesses, such as those used for cardiac catheterization (e.g., usually 4-8 Fr).

### BRIEF SUMMARY

Vasculature closure device delivery systems, vasculature closure systems, and methods for closing a puncture site existing in a wall of a vessel are provided. According to one aspect, a vasculature closure device delivery system for intraluminally positioning a vasculature closure device against a puncture site existing in a wall of a vessel is provided. In one or more embodiments, the vasculature closure device delivery system includes a loading tube configured to receive the vasculature closure device therein, a push rod configured to extend through the loading tube and to push the vasculature closure device out of the loading tube and into a lumen of the vessel, and an automatic deployment mechanism configured to detect when the vasculature closure device is positioned at the puncture site and to automatically deploy the vasculature closure device thereabout.

In some embodiments, the automatic deployment mechanism comprises a handle and a spring mechanism, wherein a first end of the spring mechanism is connected to the push rod, and wherein a second end of the spring mechanism is connected to the handle. At least a portion of the push rod may be received within the handle, and the handle and the push rod may be configured to move axially with respect to one another from a pre-deployment configuration to a deployment configuration. The spring mechanism may be configured to resist axial movement of the handle and the push rod from the pre-deployment configuration to the deployment configuration. The spring mechanism may include one of an extension spring, a compression spring, a constant force coil spring, and a friction element. In some embodiments, the automatic deployment mechanism also includes a lock mechanism configured to selectively lock the handle and the push rod in the pre-deployment configuration. The lock mechanism may include a locking pin configured to selectively extend through the handle and engage a portion of the push rod.

In some embodiments, the vasculature closure device delivery system also includes an implant holder configured to hold the vasculature closure device for delivery into the vessel, a restraining thread configured to maintain the vasculature closure device in a collapsed configuration during delivery into the vessel, and a release wire configured to release the vasculature closure device from the implant holder and the restraining thread, such that the vasculature closure device is allowed to expand from the collapsed configuration into an expanded configuration. A first end of the release wire may be connected to the handle, and a second end portion of the release wire may engage the implant holder and the restraining thread. In some embodiments, the release wire is configured to release the vasculature closure device from the implant holder and the restraining thread upon movement of the handle and the push rod from the pre-deployment configuration to the deployment configuration. In some embodiments, the release wire is configured to release the vasculature closure device from the implant holder and the restraining thread when a resistance force provided by the vasculature closure device positioned at the puncture site is greater than a spring force provided by the spring mechanism.

In some embodiments, the vasculature closure device delivery system also includes a resistance element configured to resist removal of the vasculature closure device prior to expansion of the vasculature closure device. The release wire may be configured to release the vasculature closure device from the implant holder and the restraining thread when a sum of a resistance force provided by the resistance element and a resistance force provided by the vasculature closure device positioned at the puncture site is greater than a spring force provided by the spring mechanism. The resistance element may include a wire extending over a distal portion of the vasculature closure device. In some embodiments, the vasculature closure device delivery system also includes a resistance element restraining thread configured to maintain the resistance element in a constrained configuration prior to release of the vasculature closure device, and allow the resistance element to move to an unconstrained configuration upon release of the vasculature closure device. The resistance element may be configured to provide a first resistance force when in the constrained configuration and a second resistance force when in the unconstrained configuration, wherein the first resistance force is greater than the second resistance force. The resistance element may be configured to provide a resistance force that varies during delivery of the vasculature closure device.

According to another aspect, a vasculature closure system for closing a puncture site existing in a wall of a vessel is provided. In one or more embodiments, the vasculature closure system includes a vasculature closure device and a vasculature closure device delivery system. The vasculature closure device may include an expandable support frame deployable within the vessel, and a sealing membrane at least partially supported by the support frame, wherein the vasculature closure device is configured to expand from a collapsed configuration into an expanded configuration to intraluminally position the sealing membrane against the puncture site.

According to a further aspect, a method for closing a puncture site existing in a wall of a vessel is provided. In one or more embodiments, the method includes deploying a vasculature closure device intraluminally within the vessel via a vasculature closure device delivery system, and positioning a sealing membrane of the vasculature closure device against the puncture site.

According to still another aspect, a vasculature closure device delivery system for intraluminally positioning a vasculature closure device against a puncture site existing in a wall of a vessel is provided. In one or more embodiments, the vasculature closure device delivery system includes a loading tube configured to receive the vasculature closure device therein, a push rod configured to extend through the loading tube and to push the vasculature closure device out of the loading tube and into a lumen of the vessel, a handle configured to receive at least a portion of the push rod therein, and a spring mechanism comprising a first end connected to the push rod and a second end connected to the handle.

### BRIEF DESCRIPTION OF THE DRAWINGS

FIG. 1 is a partial cross-sectional side view of an example vasculature closure device (VCD) delivery system for delivering and securing a VCD within a vessel according to one or more embodiments of the disclosure.

FIG. 2A is a cross-sectional view of an example automatic deployment mechanism of a VCD delivery system according to one or more embodiments of the disclosure, showing a handle and a push rod of the VCD delivery system in a locked, pre-deployment configuration.

FIG. 2B is a cross-sectional view of the automatic deployment mechanism of FIG. 2A, showing the handle and the push rod in an unlocked, deployment configuration.

FIG. 3A is a cross-sectional view of an example automatic deployment mechanism of a VCD delivery system according to one or more embodiments of the disclosure, showing a handle and a push rod of the VCD delivery system in a locked, pre-deployment configuration.

FIG. 3B is a cross-sectional view of an example automatic deployment mechanism of a VCD delivery system according to one or more embodiments of the disclosure, showing a handle and a push rod of the VCD delivery system in a locked, pre-deployment configuration.

FIG. 4A is a partial cross-sectional side view of an example VCD delivery system for delivering and securing a VCD within a vessel according to one or more embodiments of the disclosure, showing a distal element of the VCD delivery system in a coupled, constrained configuration.

FIG. 4B is a partial cross-sectional side view of the VCD delivery system of FIG. 4A, showing the distal element in an uncoupled, unconstrained configuration.

### DETAILED DESCRIPTION

Vasculature closure devices (VCDs) and systems and methods for their use are provided to address some or all of the above-described needs. In particular, VCDs, VCD delivery systems, and VCD delivery methods that quickly and effectively close openings in vessel walls have been developed. Such VCDs, systems, and methods advantageously may avoid, or at least substantially reduce, the time and expense of applying manual pressure to an opening, simplify the steps required to close the opening, avoid widening of the opening, and more effectively retain the VCD in the vessel.

The VCDs, systems, and methods provided herein generally may be configured in a manner similar to the VCDs, systems, and methods provided in International Patent Application Publication No. WO 2011/046932 to Penner et al. and International Patent Application Publication No. WO 2013/188351 to Penner et al. (which are referred to herein as “the prior publications”), as will be appreciated upon review of the following description. In the interest of conciseness of the present disclosure, similarities in structure, function, and method steps of the VCDs, systems, and methods provided herein and those of the prior publications are not repeated in this disclosure. Where applicable, similar features of the subject matter of the present disclosure and the prior publications generally are described using similar or identical terminology and may be shown using similar or identical reference numbers. Certain differences in structure, function, and method steps of the VCDs, systems, and methods provided in the present disclosure and those of the prior publications are described herein below in detail.

A VCD, according to various embodiments described herein, includes at least one sealing membrane and at least one expandable support frame attached to, integrated with, or otherwise supporting the sealing membrane. The support frame is utilized to expand the sealing membrane, and the overall VCD, from a collapsed configuration to an expanded configuration when deployed within a vessel. For example, the VCD may be configured for rolling into the collapsed configuration for delivery into a vessel and for unrolling into the expanded configuration for securing the VCD within the vessel. The support frame may be configured such that it expands enough to force the sealing membrane to move into a position against a vessel puncture. The pressure exerted by the support frame may vary but is effective to at least partially maintain the VCD at the desired position within the vessel and to at least partially press the sealing membrane against the vessel puncture. Upon positioning the VCD and exerting pressure by the sealing membrane against the vessel puncture, blood leakage is prevented or significantly reduced, and hemostasis and healing are promoted. In some instances, the sealing membrane of the VCD may significantly reduce blood leakage from the vessel puncture, while complete hemostasis is achieved by a thrombus formed on or around the sealing membrane against the puncture. Thrombus forming capabilities may be enhanced by providing thrombus promoting materials on the sealing membrane and/or a tether, positioning tab, or anchoring tab of the VCD. The VCD may be left in the secured position within the vessel for essentially any period of time, which may be indefinitely in certain embodiments.

The VCD may be used to close punctures or penetrations in vessels in human or other animals (e.g., mammalian). Such an animal may be referred to herein as a patient. As used herein, the term “vessel” refers to arteries, veins, other vascular lumens for carrying blood or lymph, or other body lumens, such as, but not limited to, body lumens of the gastrointestinal system (e.g., the esophagus, the stomach, the small intestine, or the large intestine), the airway system (e.g., the trachea, the bronchus, or the bronchioles), the urinary system (e.g., the bladder, the ureters, or the urethra), or the cerebrospinal system (e.g., subarachnoid space or the ventricular system around and/or inside the brain and/or the spinal cord). The VCD may be dimensioned for effective use with a variety of vessel anatomies and sizes in adult and pediatric patients, as well as with punctures at a variety of vessel sites within the patient. It is envisioned that the VCD may be adapted for use in closing punctures in other body lumens in conjunction with various surgical procedures. For example, in one other embodiment, the VCD may be adapted for use to close lumen punctures during natural orifice transluminal endoscopic surgery or to close a lumbar puncture. Various embodiments of VCDs and features thereof are described in detail in the prior publications.

In certain embodiments, the VCD delivery systems and the VCD delivery methods provided herein facilitate detection of a puncture site in a wall of a vessel and automatic deployment of a VCD when the VCD is positioned at the puncture site. In particular, the VCD delivery systems advantageously may include an automatic deployment mechanism configured to detect when the VCD is positioned at the puncture site and to automatically deploy the VCD thereabout, and the VCD delivery methods advantageously may include automatically deploying the VCD upon detecting that the VCD is positioned at the puncture site.

In certain embodiments, the VCD delivery systems and the VCD delivery methods provided herein increase resistance of a VCD positioned at a puncture site in a wall of a vessel and thus increase tactile feedback to a physician or a force applied to an automatic deployment mechanism, thereby facilitating reliable detection of the puncture site and deployment of the VCD. In particular, the VCD delivery systems advantageously may include a resistance element configured to resist removal of the VCD from the vessel prior to expansion of the VCD therein, and the VCD delivery methods advantageously may include providing increased resistance to removal of the VCD from the vessel prior to expansion of the VCD therein.

### VCD Delivery Systems and Methods Including an Automatic Deployment Mechanism

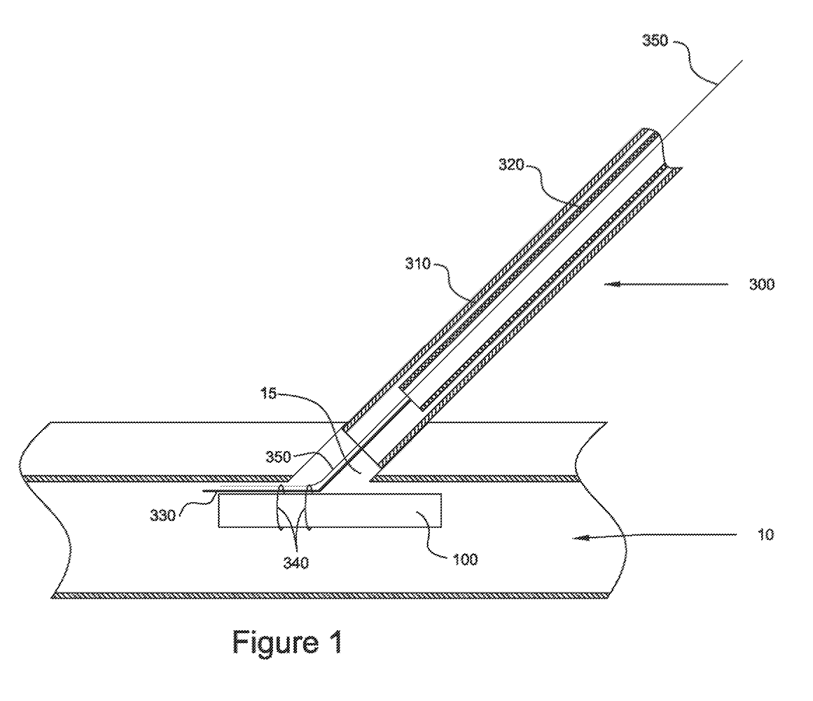

Referring to the figures, FIG. 1 depicts an example VCD delivery system 300 configured for implanting a vasculature closure device (VCD) 100 intraluminally within a patient's vessel 10. Specifically, the VCD delivery system 300 may be used to deliver the VCD 100 into the vessel 10 through a puncture site 15 (which is interchangeably referred to herein as an “access hole,” “access site,” “vessel puncture,” “puncture hole,” “vessel puncture site,” or other similar variations thereof) existing in a wall of the vessel 10, to intraluminally position the VCD 100 against the puncture site 15, and to facilitate deployment of the VCD 100 about the puncture site 15. For example, after delivering the VCD 100 into the vessel 10, the VCD delivery system 300 or a portion thereof may be retracted to position the VCD 100 against the puncture site 15 for subsequent expansion thereabout. As described in the prior publications, upon deployment, the VCD 100 may be secured within the vessel 10 to at least temporarily seal a target area at or near the puncture site 15.

According to the illustrated example, the VCD delivery system 300 includes a loading tube 310, a push rod 320, an implant holder 330, a restraining thread 340, and a release wire 350, which generally may be configured and used in a manner similar to corresponding features described in the prior publications. In particular, the loading tube 310 may be configured to at least partially receive the VCD 100 in a collapsed configuration and to guide the collapsed VCD 100 through tissue to or through the puncture site 15. The push rod 320 may be configured to extend through the loading tube 310 and to push the collapsed VCD 100 out of the loading tube 310 and expose the VCD 100 within a lumen of the vessel 10. The implant holder 330 may be configured to hold the collapsed VCD 100 and to guide the collapsed VCD 100 through the puncture site 15 and within the vessel 10, and the restraining thread 340 may be configured to maintain the VCD 100 in the collapsed configuration during delivery into the vessel 10 and prior to expansion of the VCD 100 within the vessel 10. The release wire 350 may be configured to liberate the restraining thread 340 and to release the VCD 100 from the implant holder 330 and the restraining thread 340 such that the VCD 100 may expand from the collapsed configuration into an expanded configuration within the vessel 10.

Referring to FIG. 1, a crucial point during the VCD 100 delivery procedure is detecting the puncture site 15 in the wall of the vessel 10 (after the VCD 100 has been delivered into the vessel 10 and exposed therein). In some embodiments of the VCD delivery method, during retraction of the VCD delivery system 300, the physician tactilely feels the resistance of the VCD 100 at the puncture site 15 and then deploys the VCD 100 (i.e., expands or allows the VCD 100 to expand) by pulling the release wire 350, thereby liberating the VCD restraining thread 340. An improvement to the VCD delivery system 300 would be to include a mechanism configured to detect when the VCD 100 is positioned at the puncture site 15 and to deploy the VCD 100 automatically without physician interaction (i.e., without the physician having to determine when to selectively deploy the VCD 100).

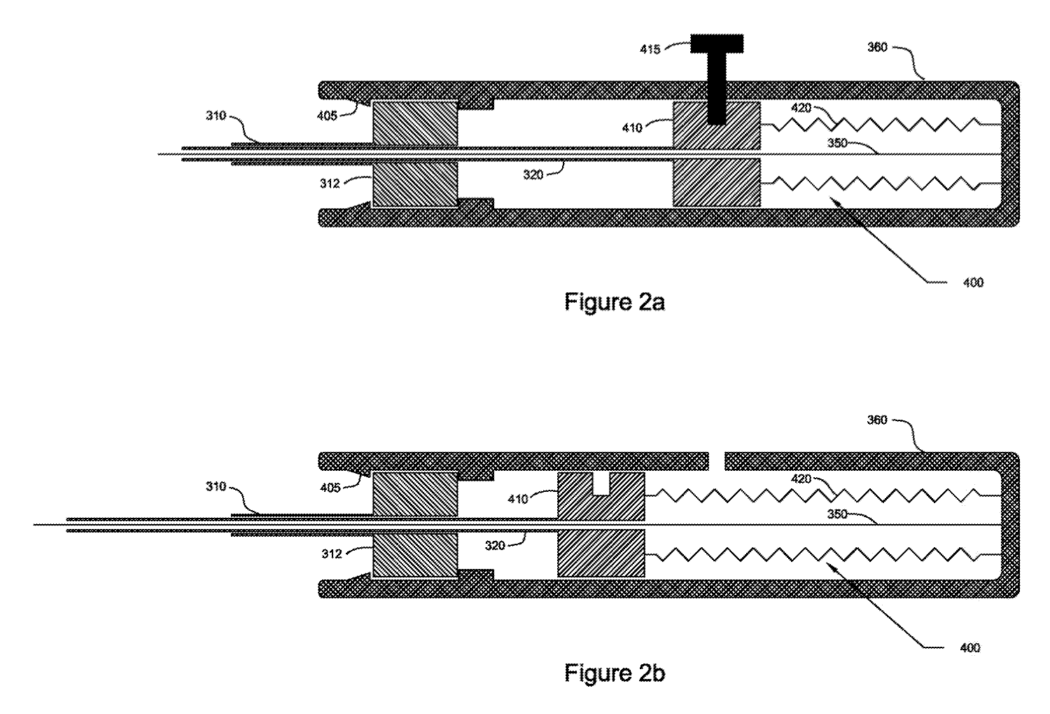

Referring to FIG. 2A, an example VCD automatic deployment mechanism 400 of the VCD delivery system 300 is illustrated. According to the illustrated example, the automatic deployment mechanism 400 includes a handle 360, a sliding ring 410, a locking pin 415, and a spring mechanism 420. The handle 360 may be configured to be grasped by a physician for guiding the VCD delivery system 300 and the attached VCD 100 to the puncture site 15 of the vessel 10. At the distal end of the push rod 320, the VCD 100 is assembled, and at the proximal end, the push rod 320 is connected via the spring mechanism 420 to the VCD delivery system handle 360. The spring mechanism 420 may be an extension spring, as shown, although other types of springs may be used. At the proximal end of the push rod 320, the sliding ring 410 is rigidity attached and sized so that the sliding ring 410 freely slides within the VCD delivery system handle 360. The locking pin 415 releasably engages the sliding ring 410 and is configured to control movement of the sliding ring 410 within the handle 360. In particular, when the locking pin 415 engages the sliding ring 410, the locking pin 415 may inhibit movement of the sliding ring 410 within the handle 360, and when the locking pin 415 is released therefrom, the sliding ring 415 may freely slide within the handle 360. In this manner, the locking pin 415 and the sliding ring 410 may form a lock mechanism for selectively locking the automatic deployment mechanism 400. In some embodiments, the handle 360 and/or the push rod 320 include means for indicating the correct orientation of the VCD delivery system 300 relative to the patient. Such orientation indicators may include marks, text printing, coloring of part of the handle 360 or the push rod 320, curves, shaping of the handle 360, e.g., rounding of at least part of the handle 360 and/or forming surfaces of different radius of curvature including flat surfaces, and variation of roughness of the handle 360. In some embodiments, either or both of the sliding ring 410 and the VCD delivery system handle 360 have matching slots and/or protrusions that orientate the sliding ring 410 relative to the VCD delivery system handle 360 and prevent relative rotation thereof. In addition, the sliding ring 410 may be connected to the VCD delivery system handle 360 by the spring mechanism 420, as shown. The VCD release wire 350, which may extend from a lumen at the proximal end of the push rod 320, may be rigidity attached to the VCD delivery system handle 360.

The VCD 100 is exposed in the vessel 10 during the VCD delivery procedure when the physician orients the VCD delivery system handle 360 and keeps it stationary and pulls the loading tube 310 toward the VCD delivery system handle 360. Referring to FIG. 2A, when a proximal end 312 of the loading tube 310 is pulled into contact with the VCD delivery system handle 360, the proximal end 312 is joined/locked to the VCD delivery system handle 360 by a loading tube integration mechanism 405. The loading tube integration mechanism 405 may be integrated into the proximal end 312 of the loading tube 310, the VCD delivery system handle 360, or both, or as a separate part. In some embodiments, as shown, the loading tube integration mechanism 405 includes one or more ramped protrusions of the handle 360 configured to allow proximal movement of the proximal end 312 of the loading tube 310 into the handle 360 and to inhibit subsequent distal movement of the proximal end 312 of the loading tube 310 out of the handle 360. When the physician decides to proceed with the VCD deployment procedure, the deployment mechanism locking pin 415 is removed, after which the physician proceeds by pulling back the VCD delivery system handle 360 (i.e., pulling the handle 360 away from the vessel 10). Alternative lock mechanisms for selectively locking the automatic deployment mechanism 400, such as a release button or a rotating lock for releasably engaging the sliding ring 410, may be used. In some other embodiments, the locking pin 415 is combined within the loading tube integration mechanism 405 such that, by completing the integration of the loading tube 310 and the VCD delivery system handle 360, the locking pin 415 is automatically removed or triggered, thereby allowing the implantation procedure to continue without requiring the physician to selectively unlock the lock mechanism. Alternatively, the lock mechanism may be associated with the loading tube 310.

As the physician pulls back the VCD delivery system handle 360, the VCD 100 encounters resistance at the puncture site 15 and the push rod 320 remains stationary while the VCD delivery system handle 360 continues to travel. Because the push rod 320, via the sliding ring 410, is also connected to the VCD delivery system handle 360 by the spring mechanism 420, a force is applied to the sliding ring 410, pulling it by the calibrated force of the spring mechanism 420. When the resistance force created by the VCD 100 at the puncture site 15 is greater than the calibrated force exerted by the spring mechanism 420, the release wire 350 moves relative to the push rod 320, as shown in FIG. 2B, until the VCD 100 is deployed (i.e., expanded or allowed to expand). Other types of springs or systems (e.g. elastomeric, pneumatic) that provide a regulated force over a distance of travel may be used. In such a manner, the physician is not required to tactilely feel the positioning of the VCD 100 at the puncture site 15, and the deployment procedure can be accomplished with less X Ray radiation, faster, with less physician training, and with a higher and consistent success rate.

In addition to the force exerted by the spring mechanism 420, friction forces generated between the VCD release wire 350 and the VCD restraining thread 340, between the VCD release wire 350 and the lumen of the push rod 320, and/or between the VCD release wire 350 and the implant holder 330 can increase the force required to pull the VCD release wire 350 back via the handle 360. In this manner, the VCD restraining thread 340, the push rod 320, and/or the implant holder 330 may function as a friction element. Additionally or alternatively, the VCD delivery system 300 may include one or more other friction elements configured to frictionally engage the VCD release wire 350 as the wire 350 is pulled back via the handle 360. In some embodiments, the inherent force required to pull the VCD release wire 350 (i.e., the force required due to one or more of the foregoing friction forces) may be sufficient such that the spring mechanism 420 is not required. In addition, certain features, such as component roughness or VCD release wire 350 path tortuosity, may be used to increase or decrease the force required to pull the VCD release wire 350.

In some embodiments, the loading tube 310 is substantially rigid. For example, the loading tube 310 may be sufficiently rigid and have sufficient strength to prevent deformation or kinking of the loading tube 310 during use of the VCD delivery system 300. In some embodiments, the push rod 320 also is substantially rigid. For example, the push rod 320 may be sufficiently rigid and have sufficient strength to prevent deformation or kinking of the push rod 320 during use of the VCD delivery system 300. As shown in FIG. 1, an outer diameter of the push rod 320 may be less than an inner diameter of the loading tube 310. In this manner, the rigid loading tube 310 and the rigid push rod 320 may be configured such that no friction force is generated therebetween when the push rod 320 moves relative to the loading tube 310. Accordingly, premature release of the VCD release wire 350 (i.e., prior to the VCD 100 being positioned at the puncture site 15), due to interaction between the loading tube 310 and the push rod 320, may be avoided.

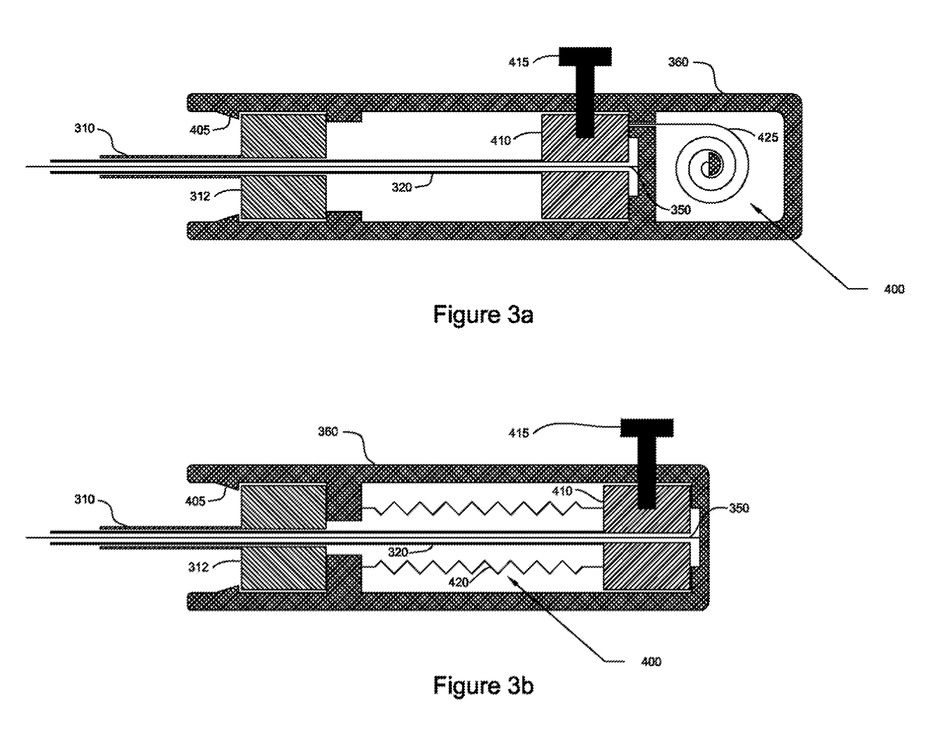

Referring to FIG. 3A, another example VCD automatic deployment mechanism 400 of the VCD delivery system 300 is illustrated. According to the illustrated example, the spring mechanism 420 is a constant force spring 425 connecting the sliding ring 410 to the VCD delivery system handle 360.

Referring to FIG. 3B, another example VCD automatic deployment mechanism 400 of the VCD delivery system 300 is illustrated. According to the illustrated example, the spring mechanism 420 is a compression spring connecting the sliding ring 410 to the VCD delivery system handle 360.

Ultimately, the spring mechanism 420, which may be an extension spring, a compression spring, a constant force coil spring, or other type of spring, can be assembled with an initial preload force. The initial preload force of the spring mechanism 420 may be equal to the VCD release force (i.e., the force required to deploy the VCD 100), such that when the resistance to retracting the VCD 100 through the vessel puncture site 15 exceeds the initial preload force, the release wire 350 moves relative to the push rod 320 until the VCD 100 is deployed (i.e., expanded or allowed to expand) within the vessel 10.

### VCD Delivery Systems and Methods Providing Increased VCD Retraction Force

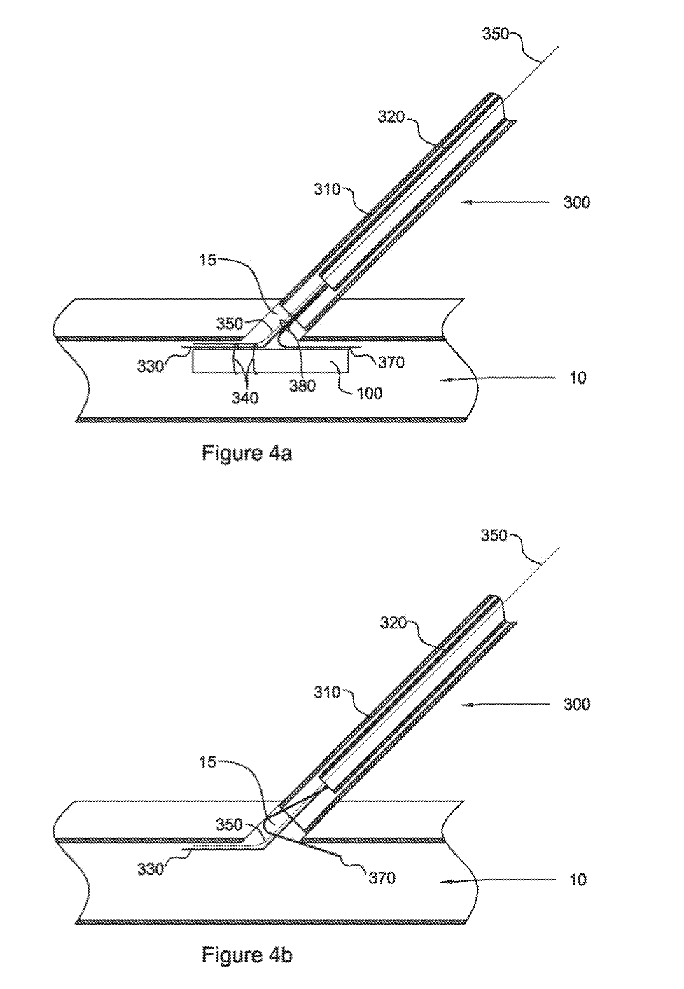

In certain applications, it may be advantageous to increase the resistance of the VCD 100 at the puncture site 15 and therefore increase the tactile feedback to the physician or the force applied to the automatic deployment mechanism 400, so that the identification of the puncture site 15 and deployment of the VCD 100 is more reliable. FIGS. 4A and 4B depict another example VCD delivery system 300 configured for implanting a VCD 100 intraluminally within a patient's vessel 10. The VCD delivery system 300 includes a mechanism configured to increase the resistance force when the VCD 100 is positioned at the puncture site 15. According to the illustrated example, the VCD delivery system 300 includes a stiff distal element 370 (which is interchangeably referred to herein as a “resistance element”). The distal element 370 may be a part of the implant holder 330 or may be a separate component attached to or positioned adjacent the implant holder 330. The length of the distal element 370 may be configured to be the same as, longer than, or shorter than the distal end of the VCD 100 when the VCD 100 is attached to the VCD delivery system 300. The distal element 370 may be formed from a wire or may be manufactured by laser cutting, chemical etching, water-jet cutting, milling, and/or using electrical arc processes. The manufacturing and shaping of the distal element 370 may be carried out in a separate process or in the same process as the manufacturing and shaping of the implant holder 330. Suitable processes may include thermal setting, plastic deformation, casting, and/or any other means for shaping materials.

In some embodiments, the distal element 370 has a variable rigidity such that, prior to deployment of the VCD 100, the distal element 370 significantly increases the force to retract the VCD 100, while after deployment of the VCD 100, the rigidity of the distal element 370 is significantly reduced so that the distal element 370 does not damage the vessel 10 when the VCD delivery system 300 is removed from the vessel 10.

In some embodiments, the distal element 370 is formed of or includes a superelastic Nitinol wire which is coupled to the implant holder 330 by a distal element restraining thread 380, as shown. Similar to the VCD restraining thread 340, the release wire 350 also may secure the distal element restraining thread 380. In this manner, when the release wire 350 is pulled, the VCD restraining thread 340 and distal element restraining thread 380 may be liberated, thereby deploying the VCD 100 and uncoupling the distal element 370. Referring to FIG. 4B, once the distal element 370 is uncoupled from the implant holder 330, the distal element 370 may be free to move or rotate relative to the implant holder 330. In this manner, the distal element 370 may be retracted through the puncture site 15 with greatly reduced resistance, without the fear of damaging the vessel 10.

It is appreciated that many modifications and variations of the devices, systems, and methods described herein, such as dimensional, size, and/or shape variations, will be apparent to those skilled in the art from the foregoing detailed description. Such modifications and variations are intended to come within the scope of the appended claims.

1-22. (canceled)

23. A vasculature closure device delivery system for intraluminally positioning an expandable vasculature closure device against a puncture site existing in a wall of a vessel, the vasculature closure device delivery system comprising:
a loading tube configured to receive the vasculature closure device therein;
a push rod configured to extend through the loading tube, wherein the loading tube and the push rod are configured to move with respect to one another to expose the vasculature closure device within a lumen of the vessel; and
an automatic deployment mechanism configured to detect when the vasculature closure device is positioned within the lumen at the puncture site and to automatically allow the vasculature closure device to expand within the lumen about the puncture site.

24. The vasculature closure device delivery system of claim 23, wherein the automatic deployment mechanism comprises a handle configured to receive at least a portion of the push rod therein, and wherein the handle and the push rod are configured to move axially with respect to one another from a pre-deployment configuration to a deployment configuration.

25. The vasculature closure device delivery system of claim 24, wherein the automatic deployment mechanism further comprises a spring mechanism configured to resist axial movement of the handle and the push rod from the pre-deployment configuration to the deployment configuration.

26. The vasculature closure device delivery system of claim 25, wherein the spring mechanism comprises a first end connected to the push rod and a second end connected to the handle.

27. The vasculature closure device delivery system of claim 25, wherein the spring mechanism comprises one of an extension spring, a compression spring, a constant force coil spring, and a friction element.

28. The vasculature closure device delivery system of claim 25, wherein the automatic deployment mechanism further comprises a lock mechanism configured to selectively lock the handle and the push rod in the pre-deployment configuration.

29. The vasculature closure device delivery system of claim 25, further comprising:
an implant holder configured to hold the vasculature closure device for delivery into the vessel;
a restraining thread configured to maintain the vasculature closure device in a collapsed configuration during delivery into the vessel; and
a release wire configured to release the vasculature closure device from the implant holder and the restraining thread, such that the vasculature closure device is allowed to expand from the collapsed configuration into an expanded configuration within the lumen about the puncture site.

30. The vasculature closure device delivery system of claim 29, wherein a first end of the release wire is connected to the handle, wherein a second end portion of the release wire engages the implant holder and the restraining thread, and wherein the release wire is configured to release the vasculature closure device from the implant holder and the restraining thread upon movement of the handle and the push rod from the pre-deployment configuration to the deployment configuration.

31. The vasculature closure device delivery system of claim 29, wherein the release wire is configured to release the vasculature closure device from the implant holder and the restraining thread when a resistance force provided by the vasculature closure device positioned within the lumen at the puncture site is greater than a spring force provided by the spring mechanism.

32. The vasculature closure device delivery system of claim 29, further comprising a resistance element configured to resist removal of the vasculature closure device from the lumen prior to expansion of the vasculature closure device, wherein the release wire is configured to release the vasculature closure device from the implant holder and the restraining thread when a sum of a resistance force provided by the resistance element and a resistance force provided by the vasculature closure device positioned within the lumen at the puncture site is greater than a spring force provided by the spring mechanism.

33. The vasculature closure device delivery system of claim 32, further comprising a resistance element restraining thread configured to maintain the resistance element in a constrained configuration prior to release of the vasculature closure device and to allow the resistance element to move to an unconstrained configuration upon release of the vasculature closure device, wherein the resistance element is configured to provide a first resistance force when in the constrained configuration and a second resistance force when in the unconstrained configuration, and wherein the first resistance force is greater than the second resistance force.

34. The vasculature closure device delivery system of claim 24, further comprising a release wire connected to the handle and configured to release the vasculature closure device from the vasculature closure device delivery system upon movement of the handle and the push rod from the pre-deployment configuration to the deployment configuration, such that the vasculature closure device is allowed to expand from a collapsed configuration into an expanded configuration within the lumen about the puncture site.

35. A method for closing a puncture site existing in a wall of a vessel, the method comprising:
positioning an expandable vasculature closure device within a loading tube of a delivery system, wherein the vasculature closure device comprises an expandable support frame and a sealing membrane at least partially supported by the support frame;
moving the loading tube and a push rod of the delivery system with respect to one another to expose the vasculature closure device within a lumen of the vessel;
detecting that the vasculature closure device is positioned within the lumen at the puncture site, via an automatic deployment mechanism of the delivery device; and
automatically allowing the vasculature closure device to expand within the lumen about the puncture site, via the automatic deployment mechanism.

36. The method of claim 35, wherein the automatic deployment mechanism comprises a handle configured to receive at least a portion of the push rod therein, and wherein automatically allowing the vasculature closure device to expand within the lumen about the puncture site comprises moving the handle and the push rod axially with respect to one another from a pre-deployment configuration to a deployment configuration.

37. The method of claim 36, wherein the automatic deployment mechanism further comprises a spring mechanism, and wherein moving the handle and the push rod axially with respect to one another from the pre-deployment configuration to the deployment configuration comprises loading the spring mechanism.

38. The method of claim 37, wherein the spring mechanism comprises one of an extension spring, a compression spring, a constant force coil spring, and a friction element.

39. The method of claim 37, wherein the delivery system further comprises a release wire connected to the handle, and wherein automatically allowing the vasculature closure device to expand within the lumen about the puncture site further comprises releasing the vasculature closure device from the delivery system via the release wire, such that the vasculature closure device is allowed to expand from a collapsed configuration into an expanded configuration within the lumen about the puncture site.

40. The method of claim 39, wherein the release wire releases the vasculature closure device from the delivery system when a resistance force provided by the vasculature closure device positioned within the lumen at the puncture site is greater than a spring force provided by the spring mechanism.

41. A vasculature closure system for closing a puncture site existing in a wall of a vessel, the vasculature closure system comprising:
an expandable vasculature closure device comprising:
an expandable support frame deployable within a lumen of the vessel; and
a sealing membrane at least partially supported by the support frame;
wherein the vasculature closure device is configured to expand from a collapsed configuration into an expanded configuration within the lumen of the vessel; and
a vasculature closure device delivery system comprising:
a loading tube configured to receive the vasculature closure device therein;
a push rod configured to extend through the loading tube, wherein the loading tube and the push rod are configured to move with respect to one another to expose the vasculature closure device within the lumen of the vessel; and
an automatic deployment mechanism configured to detect when the vasculature closure device is positioned within the lumen at the puncture site and to automatically allow the vasculature closure device to expand from the collapsed configuration into the expanded configuration within the lumen about the puncture site.

42. A vasculature closure device delivery system for intraluminally positioning an expandable vasculature closure device against a puncture site existing in a wall of a vessel, the vasculature closure device delivery system comprising:
a loading tube configured to receive the vasculature closure device therein;
a push rod configured to extend through the loading tube, wherein the loading tube and the push rod are configured to move with respect to one another to expose the vasculature closure device within a lumen of the vessel;
a handle configured to receive at least a portion of the push rod therein, wherein the handle and the push rod are configured to move axially with respect to one another from a pre-deployment configuration to a deployment configuration;
a spring mechanism configured to resist axial movement of the handle and the push rod from the pre-deployment configuration to the deployment configuration; and
a release wire connected to the handle and configured to release the vasculature closure device from the vasculature closure device delivery system upon movement of the handle and the push rod from the pre-deployment configuration to the deployment configuration.
